SEQUENCE LISTING (1 sequence)

SEQ ID NO: 1 - protein, 369 aa, Mus musculus
MVPCTLLLLLAAALAPTQTRAGPHSLRYFVTAVSRPGLGEPRYMEVGYVDDTEFVRFDSDAENPRYEPRARWMEQEGPEYWERETQKAKGNEQSFRVDLRTLLGYYNQSKGGSHTIQVISGCEVGSDGRLLRGYQQYAYDGCDYIALNEDLKTWTAADMAALITKHKWEQAGEAERLRAYLEGTCVEWLRRYLKNGNATLLRTDSPKAHVTHHSRPEDKVTLRCWALGFYPADITLTWQLNGEELIQDMELVETRPAGDGTFQKWASVVVPLGKEQYYTCHVYHQGLPEPLTLRWEPPPSTVSNMATVAVLVVLGAAIVTGAVVAFVMKMRRRNTGGKGGDYALAPGSQTSDLSLPDCKVMVHDPHSLA

(19) United States
(12) Patent Application Publication
(10) Pub. No.: US 2022/0091103 A1
(43) Pub. Date: Mar. 24, 2022

(54) VITRO ASSAY FOR DRUG DISCOVERY OF VALVULAR HEART DISEASE

(71) Applicant: UNIVERSITY OF SOUTH CAROLINA, COLUMBIA, SC (US)

(72) Inventor: Mohamad AZHAR, Irmo, SC (US)

(73) Assignee: UNIVERSITY OF SOUTH CAROLINA, COLUMBIA, SC (US)

(21) Appl. No.: 17/474,178

(22) Filed: Sep. 14, 2021

Related U.S. Application Data
(60) Provisional application No. 63/077,837, filed on Sep. 14, 2020.

(51) Int. Cl. G01N 33/50

(52) U.S. Cl. CPC G01N 33/5044; G01N 2500/10; G01N 2500/02; G01N 33/5014

(57) ABSTRACT

Described herein are assays, methods of use, and novel, stable lines of valve interstitial cells to provide a cell culture-based assay that can be used to quickly identify novel and potential drugs that can used for medical treatment of valve disease since potential therapeutic molecules from mouse VIC assays can be tested in genetic and experimental mouse models in preclinical studies.

### TECHNICAL FIELD

The subject matter disclosed herein is generally directed to assays, methods of use, and novel, stable lines of valve interstitial cells to provide a cell culture-based assay that can be used to quickly identify novel and potential drugs for medical treatment of valve disease as potential therapeutic molecules from mouse VIC assays can be tested in genetic and experimental mouse models in preclinical studies.

### BACKGROUND

Heart valve disease is the third leading cause of heart disease. Calcification of aortic valve and mitral valve is a major heart valve disease. There is no medical treatment and surgical valve replacement is the only option to save patient lives. Unfortunately, replaced valves could further calcify, requiring further valve replacement surgery.

Valve disease is the third most common cardiovascular disease after hypertension and coronary vascular diseases. It affects more than 5 million people in the US. It is a leading cause of valve replacement surgery with more than 100,000 valve replacements per year in the U.S. No nonsurgical medical treatment exists, and without surgery, valve stenosis leads to heart failure or death.

Valve interstitial cells (VIC) are the major resident cell type of heart valves, including aortic and mitral valves. Calcification of the aortic valve and mitral valve occurs in patients with calcific aortic valve disease and mitral valve disease, respectively. There is no medical treatment for aortic or mitral valve disease. Surgical valve replacement is the only treatment option available to the patients of valve disease. Valve calcification contributes to valve stenosis, which eventually leads to heart failure.

Discovery of drugs to inhibit VIC calcification is hindered because there no robust mouse models and in vitro cell culture. Currently, porcine and sheep VIC cells are used in in vitro valve calcification assays.

Currently, there is no in vitro mouse VIC calcification assays. Accordingly, it is an object of the present disclosure to provide a cell culture based assay that can be used to quickly identify novel and potential drugs that can used for medical treatment of valve disease. It is important to have mouse VIC calcification assays since potential therapeutic molecules emerged from mouse VIC assays can be tested in genetic and experimental mouse models in preclinical studies. The current disclosure provides a novel mouse VIC assay to identify potential drugs which could inhibit valve calcification.

Citation or identification of any document in this application is not an admission that such a document is available as prior art to the present disclosure.

### SUMMARY

The above objectives are accomplished according to the present disclosure by providing in one embodiment, a cell culture-based valve calcification assay. The assay may include at least one valve interstitial cell that may be derived from at least one Ts(H2-K1-tsA58) mouse, wherein at least one compound is exposed to the at least one valve interstitial cell, and evaluating the at least one valve interstitial cell to determine if the at least one compound inhibited osteogenic differentiation or calcification in the at least one valve interstitial cell. Further, osteogenic differentiation may be promoted via separating at least two valve interstitial cells onto at least two separate media wherein at least one media is osteogenic. Still, evaluating occurs via staining the at least one valve interstitial cell. Yet again, an extent of staining may quantified to evaluate effectiveness of the at least one compound. Moreover, the assay may be used to identify at least one molecular mechanism or pathway involved in the pathogenesis of valvular heart disease including aortic valve stenosis, myxomatous mitral valve disease, or mitral valve prolapse. Still yet, the at least one valve interstitial cell may exhibit a pathological process involved in aortic valve and mitral valve calcification.

In a further embodiment, a method for identifying new compounds for treatment of valve calcification is provided. The method may include introducing at least one potential treatment compound to an assay, the assay may contain at least one valve interstitial cell derived from at least one mouse cell and the at least one valve interstitial cell is obtained from at least one Ts(H2-K1-tsA58) mouse and may exhibit a pathological processes causing aortic valve and mitral valve calcification, and evaluating the at least one valve interstitial cell to determine if the at least one potential treatment compound inhibited osteogenic differentiation or calcification in the at least one valve interstitial cell. Further, the at least one potential treatment compound may be a novel drug, molecule, chemical, or small molecule inhibitor. Still yet, the method may include evaluating via staining the at least one valve interstitial cell. Further yet, an extent of staining may be quantified to evaluate effectiveness of the at least one potential treatment compound. Again, the assay may be used to identify at least one molecular mechanism or pathway involved in the pathogenesis of valvular heart disease including aortic valve stenosis, myxomatous mitral valve disease, or mitral valve prolapse.

In a still yet further embodiment, a method is provided for obtaining a novel, stable line of valve interstitial cells. The method may include collecting at least one aortic valve leaflet from at least one Ts(H2-K1-tsA58) mouse, centrifuging the at least one aortic valve leaflet, digesting the at least one aortic valve leaflet to remove valvular endothelial cells, pelleting the at least one aortic valve leaflet by centrifugation, washing and repelleting the at least one aortic valve leaflet; and harvesting at least one valve interstitial cell following digestion. Still further, the valve interstitial cells of the method may allow for inducible expression of at least one thermolabile large tumor antigen. Further yet, tumorigenesis in the stable line of valve interstitial cells may be temperature dependent and tumorigenesis may be inhibited at a first select temperature but allowed at a second select temperature.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present disclosure will be obtained by reference to the following detailed description that sets forth illustrative embodiments, in which the principles of the disclosure may be utilized, and the accompanying drawings of which:

FIG. 1 shows VIC grown for 4 days at a confluency of ˜80%.

FIG. 2 shows Alizarin red staining to detect calcific cellular nodules, shown in red.

FIG. 3 shows Table 1, the experimental conditions for FIG. 1.

FIG. 4 shows photographs of calcification of VIC in osteogenic media.

FIG. 5 shows photographs of cell reactions to various potential treatment compounds.

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless specifically stated, terms and phrases used in this document, and variations thereof, unless otherwise expressly stated, should be construed as open ended as opposed to limiting. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise.

Furthermore, although items, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further embodiment includes from the one particular value and/or to the other particular value. The recitation of numerical ranges by endpoints includes all numbers and fractions subsumed within the respective ranges, as well as the recited endpoints. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosure. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, a “biological sample” may contain whole cells and/or live cells and/or cell debris. The biological sample may contain (or be derived from) a “bodily fluid”. The present disclosure encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, and cell cultures from bodily fluids. Bodily fluids may be obtained from a mammal organism, for example by puncture, or other collecting or sampling procedures.

As used herein, “agent” refers to any substance, compound, molecule, and the like, which can be administered to a subject on a subject to which it is administered to. An agent can be inert. An agent can be an active agent. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

As used herein, “active agent” or “active ingredient” refers to a substance, compound, or molecule, which is biologically active or otherwise that induces a biological or physiological effect on a subject to which it is administered to. In other words, “active agent” or “active ingredient” refers to a component or components of a composition to which the whole or part of the effect of the composition is attributed.

As used herein, “administering” refers to any suitable administration for the agent(s) being delivered and/or subject receiving said agent(s) and can be oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition to the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration routes can be, for instance, auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intraderm al, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, sub arachnoid, subconjunctival, subcutaneous, sublingual, submucosal, topical, transderm al, transmucosal, transplacental, transtracheal, transtympanic, ureteral, urethral, and/or vaginal administration, and/or any combination of the above administration routes, which typically depends on the disease to be treated, subject being treated, and/or agent(s) being administered.

As used herein, “control” can refer to an alternative subject or sample used in an experiment for comparison purpose and included to minimize or distinguish the effect of variables other than an independent variable.

The term “optional” or “optionally” means that the subsequent described event, circumstance or substituent may or may not occur, and that the description includes instances where the event or circumstance occurs and instances where it does not.

As used herein, “dose,” “unit dose,” or “dosage” can refer to physically discrete units suitable for use in a subject, each unit containing a predetermined quantity of a pharmaceutical formulation thereof calculated to produce the desired response or responses in association with its administration.

As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo.

As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed by the term “subject”.

As used herein, “substantially pure” can mean an object species is the predominant species present (i.e., on a molar basis it is more abundant than any other individual species in the composition), and preferably a substantially purified fraction is a composition wherein the object species comprises about 50 percent of all species present. Generally, a substantially pure composition will comprise more than about 80 percent of all species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single species.

As used interchangeably herein, the terms “sufficient” and “effective,” can refer to an amount (e.g. mass, volume, dosage, concentration, and/or time period) needed to achieve one or more desired and/or stated result(s). For example, a therapeutically effective amount refers to an amount needed to achieve one or more therapeutic effects.

As used herein, “tangible medium of expression” refers to a medium that is physically tangible or accessible and is not a mere abstract thought or an unrecorded spoken word. “Tangible medium of expression” includes, but is not limited to, words on a cellulosic or plastic material, or data stored in a suitable computer readable memory form. The data can be stored on a unit device, such as a flash memory or CD-ROM or on a server that can be accessed by a user via, e.g. a web interface.

As used herein, “therapeutic” can refer to treating, healing, and/or ameliorating a disease, disorder, condition, or side effect, or to decreasing in the rate of advancement of a disease, disorder, condition, or side effect. A “therapeutically effective amount” can therefore refer to an amount of a compound that can yield a therapeutic effect.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as valve calcification. The effect can be therapeutic in terms of a partial or complete cure of a disease, condition, symptom or adverse effect attributed to the disease, disorder, or condition. The term “treatment” as used herein covers any treatment of cancer and/or indirect radiation damage, in a subject, particularly a human and/or companion animal, and can include any one or more of the following: (a) preventing the disease or damage from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

As used herein, the terms “weight percent,” “wt %,” and “wt. %,” which can be used interchangeably, indicate the percent by weight of a given component based on the total weight of a composition of which it is a component, unless otherwise specified. That is, unless otherwise specified, all wt % values are based on the total weight of the composition. It should be understood that the sum of wt % values for all components in a disclosed composition or formulation are equal to 100. Alternatively, if the wt % value is based on the total weight of a subset of components in a composition, it should be understood that the sum of wt % values the specified components in the disclosed composition or formulation are equal to 100.

As used herein, “water-soluble”, generally means at least about 10 g of a substance is soluble in 1 L of water, i.e., at neutral pH, at 25° C.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present disclosure. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the disclosure. For example, in the appended claims, any of the claimed embodiments can be used in any combination.

All patents, patent applications, published applications, and publications, databases, websites and other published materials cited herein are hereby incorporated by reference to the same extent as though each individual publication, published patent document, or patent application was specifically and individually indicated as being incorporated by reference.

### Kits

Any of the interstitial cells, methods, and assays described herein can be presented as a combination kit. As used herein, the terms “combination kit” or “kit of parts” refers to the compounds, compositions, formulations, particles, cells and any additional components that are used to package, sell, market, deliver, and/or administer the combination of elements or a single element, such as the valve interstitial cells, contained therein. Such additional components include, but are not limited to, packaging, syringes, blister packages, bottles, and the like. When one or more of the compounds, compositions, formulations, particles, cells, described herein or a combination thereof (e.g., agent(s)) contained in the kit are administered simultaneously, the combination kit can contain the active agent(s) in a single formulation, such as a pharmaceutical formulation, (e.g., a tablet, liquid preparation, dehydrated preparation, etc.) or in separate formulations. When the compounds, compositions, formulations, particles, and cells described herein or a combination thereof and/or kit components are not administered simultaneously, the combination kit can contain each agent or other component in separate pharmaceutical formulations. The separate kit components can be contained in a single package or in separate packages within the kit.

In some embodiments, the combination kit also includes instructions printed on or otherwise contained in a tangible medium of expression. The instructions can provide information regarding the content of the assays, compounds and/or formulations, safety information regarding the content of the assays, compounds and formulations (e.g., pharmaceutical formulations), information regarding the dosages, indications for use, and/or recommended treatment regimen(s) for the assays, compound(s) and/or pharmaceutical formulations contained therein. In some embodiments, the instructions can provide directions and protocols for administering the assays, compounds and/or formulations described herein to a subject in need thereof. In some embodiments, the instructions can provide one or more embodiments of the methods for use or testing of compounds via the assays and interstitial cells described in greater detail elsewhere herein.

The current disclosure has established that mouse valve interstitial cells (VIC) can be cultured in tissue culture dishes and the calcification can be induced in vitro in these VIC. The disclosure employed several chemical compounds and quantified their ability to change the extent of calcification of VIC in tissue culture dishes.

This innovation will lead to the identification of novel drugs, molecules, chemicals, small molecule inhibitors, etc., for medical treatment of aortic and mitral valve calcification. Successful candidates may be tested in preclinical and clinical studies to develop novel medical treatment.

This is the first and most robust mouse valve calcification assay. It will lead to identification of novel drugs for valve disease. Large number of potential drug candidates can be screened very quickly and efficiently by using this assay. Since the assay is based on cells derived from mouse valves, the lead drugs/candidates can be tested in genetic and experimental in vivo mouse models of valve disease before initiating potential clinical trials.

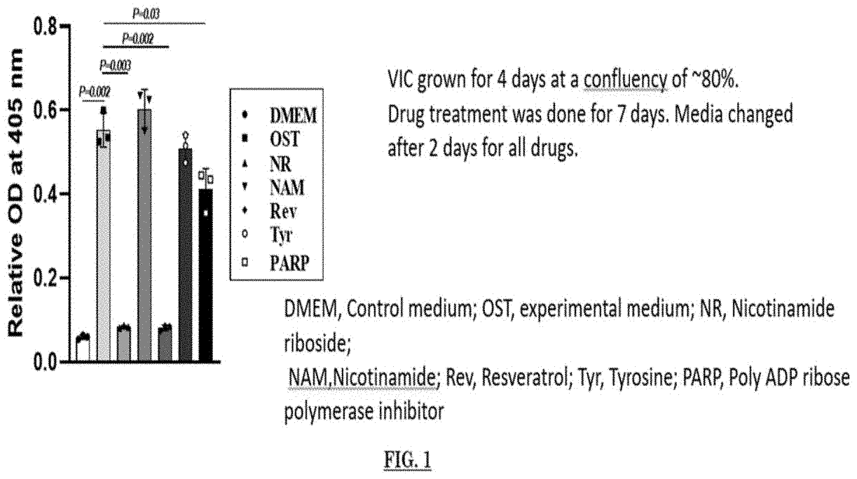

FIG. 1 shows VIC grown for 4 days at a confluency of ˜80%. Drug treatment was done for 7 days. Media changed after 2 days for all drugs. The legend indicates: DMEM, Control medium; OST, experimental medium; NR, Nicotinamide riboside; NAM, Nicotinamide; Rev, Resveratrol; Tyr, Tyrosine; PARP, and Poly ADP ribose polymerase inhibitor.

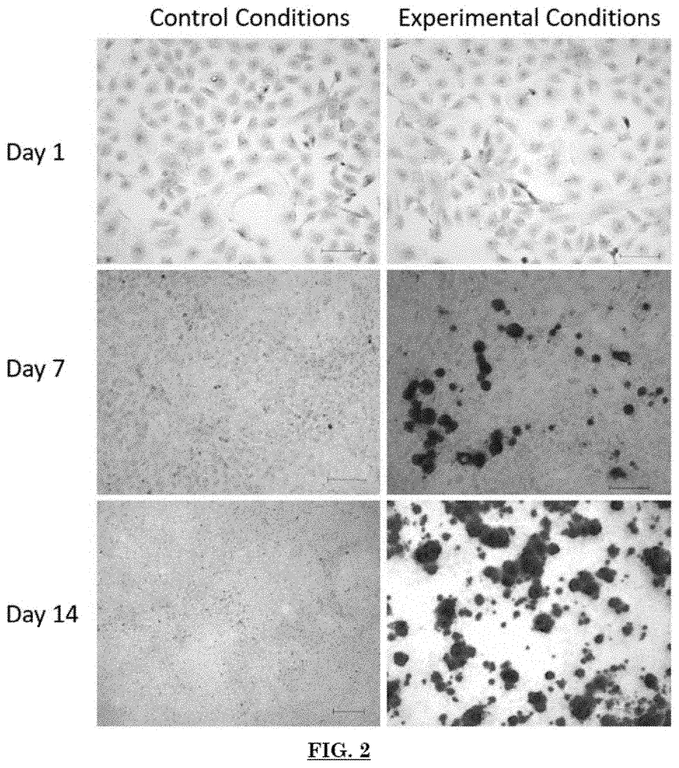

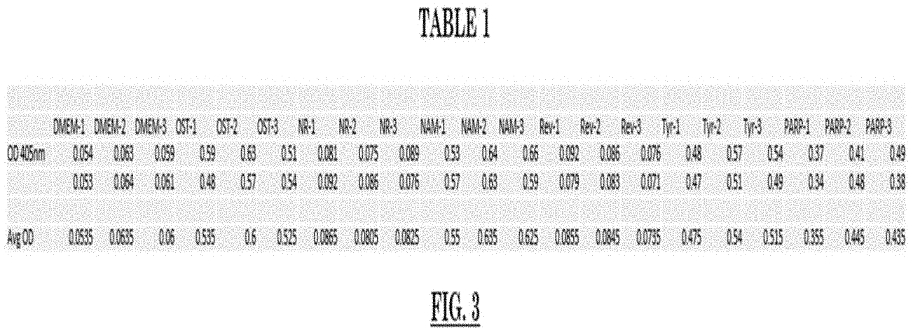

FIG. 2 shows Alizarin red staining to detect calcific cellular nodules, shown in red. FIG. 3 shows Table 1, the experimental conditions for FIG. 1.

Isolation of Mice Valve Interstitial Cell (VIC)

The valve interstitial cells (VIC) were generated from Ts(H2-K1-tsA58) mice. These mice are also known as Immortomouse and were purchased from The Jackson Laboratory (Stock No: 032619). A breeding colony of Immortomouse colony was established and bred at the Animal Resource Facility of the University of South Carolina School of Medicine. This study was approved by the Institutional Animal Care and Use Committee (IACUC) of the University of South Carolina (Protocol number: 2451-101423-042519, PI: Mohamad Azhar)). The disclosure used 4-6 weeks old Immortomouse for VIC isolation. First, mice were euthanized using isoflurane followed by cervical dislocation. Aortic valve leaflets were collected and transferred into a sterile Petri dish filled with HBSS and 1% penicillin and 1% streptomycin (GIBCO/BRL, Grand Island, N.Y., USA). All mice leaflets were stored in ice cold wash buffer; in a sterile 1.5 mL micro centrifuge tube until all leaflets were collected. At the end of tissue collection, leaflets were centrifuged at 100×g for 1 min at 4° C. Then the wash buffer was removed carefully and the leaflets were digested in 100 μl 425 U/ml collagenase II (Worthington Biochemical, LS004174) for 5 min at 37° C. to remove valvular endothelial cells (VECs).

During digestion, tissues were disrupted by gently pipetting up and down using a 200 μL pipette tip. At the end of digestion, the leaflets were pelleted by centrifugation at 100×g for 5 min and the supernatant was discarded carefully. Then aorta valve leaflets were washed twice with 500 μl wash buffer and re-pelleted the cells by centrifugation at 100×g for 5 min. Then VICs were harvested from leaflets following digestion with 100 μL of 425 U/mL collagenase II for 2 h. At the end of digestion the collagenase II solution was diluted using complete DMEM medium and centrifuged at 670×g for 5 min to pellet the VICs and remaining valve leaflet debris. Then the supernatant was discarded, and leaflets and VICs were transferred into a 24 well culture plate. VICs were grown for 5-7 days in DMEM media containing 10% FBS, 1% P/S in presence of 1×γ-IFN in a 5% CO2 incubator at 33° C., until confluency.

Effect of Drugs on Osteogenic Differentiation of VIC

The VICs were isolated from 4-6 weeks old Ts(H2-K1-tsA58) mice (The Jackson Laboratory, 600 Main Street, Bar Harbor, Me. USA 04609) aortic valves as discussed above. Cells were grown in DMEM medium supplemented with 10% fetal bovine serum (FBS) and 1% penicillin and 1% streptomycin (GIBCO/BRL, Grand Island, N.Y., USA). Cell lines were maintained in a fully-humidified incubator containing 5% CO2 at 33° C. in presence of 1×γ-IFN. The medium and FBS were purchased from Atlanta Biologicals (Norcross, Ga., USA). For osteogenic differentiation, VICs were seeded in six well plates containing 2 mL of standard DMEM media or osteogenic media (standard media supplemented with 2.6 mM Phosphate, pH 7.4). The cells were re-fed normal/osteogenic media every two or three days as necessary. At the end of 7 or 12 days of incubation/treatment osteogenic differentiation of VICs were evaluated by Alizarin Red staining. In an another set of experiments, VICs were grown for 10 days in presence or absence of several drugs and observed their efficacy in inhibiting osteogenic differentiation and calcification in VICs. We have used following drugs, Nicotinamide riboside (NR)(5 mmol/100 mL), and Nicotinamide (NAM) (5 mmol/100 mL), Resveratrol (RSV) (100 micro molar/100 mL), PARP inhibitor: Talazoparib (PARP inh)(1 micro molar/100 mL). We evaluated the calcification via Alizarin red staining as per manufacturer's protocol.

Alizarin Red Staining of VICs

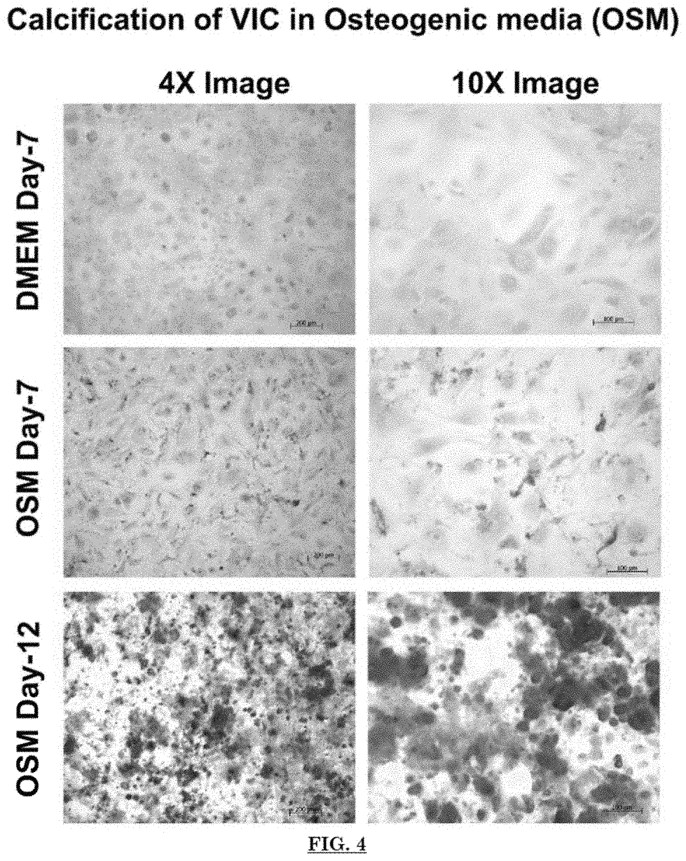

Alizarin Red staining was performed to stain the cell matrix monolayer for calcium deposits. At the end of drug treatment and incubation, the media was aspirated from the plates and phosphate buffered saline (PBS) was used to wash the cells twice. The cells were then fixed with 10% buffered formalin for 10 minutes and subsequently rinsed with PBS. The cells were then stained with Alizarin Red solution (American MasterTech Scientific, CA, USA) for 5 minutes, followed by three 1-minute rinses with distilled water. After staining, the cells were digitally photographed, see FIG. 4 showing photographs of calcification of VIC in osteogenic media (OSM).

Image Analysis Using ImageJ Software

Quantification of the degree of alizarin Red staining in digital images was performed using computer-assisted morphometric analysis software (ImageJ, NIH). For quantification of Alizarin Red staining, 4× photomicrographs were obtained using a light microscope equipped with a charge coupled device (CCD) camera attached to a PC. Photomicrographs (4×) were taken at 3 random microscopic fields and analyzed using ImageJ by thresholding for the red pixel color. The thresholding parameters were held constant for all images examined and the results were expressed as the area of the image occupied by red divided by the total area of the image. The data was then presented as the mean±S.D. of three individual values. Statistical Analysis Student t-tests were performed using SPSS Version 12.0 statistic software package. P-values<0.05 were considered statistically significant.

Original Finding and Discovery

We have generated a stable cell line of valve interstitial cells (VIC) from adult mice (FIG. 4, Top Panels at 4× and 10× magnification). This is a novel discovery. An adult mouse VIC cell line can be used for identifying molecular mechanisms and pathways involved in the pathogenesis of valvular heart disease, including aortic valve stenosis, myxomatous mitral valve disease, mitral valve prolapse).

We successfully demonstrated that osteogenic media treatment for 7-12 days induced calcification of the adult mouse VIC cell line in vitro in a petri dish (FIG. 4, Middle & Bottom Panels). Thus, the adult mouse VIC cell line is novel in vitro cell culture model to mimic the sequence of event and pathological processes involved in aortic valve (AV) and mitral valve (MV) calcification. Thus, the adult mouse VIC cell line can be used for identifying novel molecular mechanisms and pathways involved in the pathogenesis of calcific aortic valve disease, calcific aortic valve stenosis, mitral annular calcification and mitral valve calcification.

By using our novel adult mouse VIC cell line in an in vitro study, we identified four (4) candidate drugs that could be tested in small and large animals for developing potential new treatment of valvular heart disease. These four potential candidate drugs are the following:

Nicotinamide (NAM)

Nicotinamide riboside (NR)

PARP inhibitor ((Talazoparib)

Resveratrol (RSV)

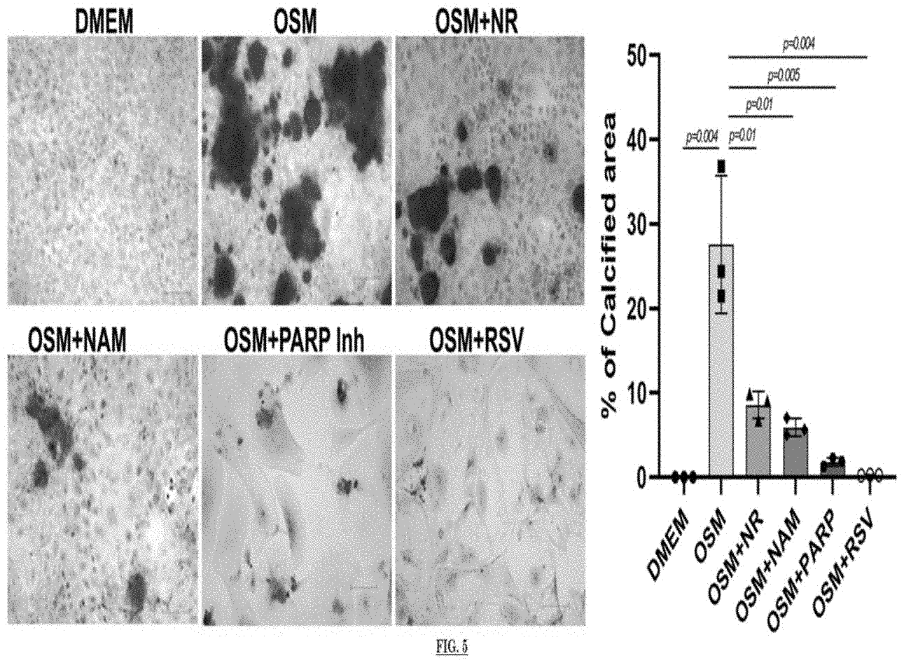

Our data specifically showed that all of these potential drugs significantly blocked the calcification of VIC cell line compared to untreated control VIC cell line when these cells were grown in vitro in a petri dish in the presence of osteogenic media for 7-12 days (FIG. 5).

Overall, the adult mouse VIC cell line is useful for identifying novel drugs, and testing various small molecules and drugs in a drug discovery process for the treatment of calcific aortic valve disease, calcific aortic valve stenosis, mitral annular calcification and mitral valve calcification.

Additional Information

Immortomouse (Source: The Jackson Laboratory)

STOCK Tg(H2-K1-tsA58)6Kio/LicrmJ

Stock No: 032619|Immortomouse

The adult mouse aortic valve interstitial cell (AVIC) cell line was produced from the 2-month old adult aortic valves from the hemizygous Immortomouse animals. Genotype of mice was confirmed by PCR of tail tissue genomic DNA. Hemizygous Immortomouse mice are viable and fertile. The Immortomouse transgene (from H-2Kb-tsA58 transgenic founder line 6 (H2ts6)) allows interferon-inducible expression of a thermolabile large tumor antigen (TAg) (and the small tumor antigen) from the SV40 thermosensitive A58 (tsA58) strain directed to widespread tissues by the interferon-inducible Class I antigen promoter from the mouse H-2Kb locus. Mice display onset of thymic hyperplasia and death with earlier onset in homozygous mice than in hemizygous mice. To prevent/reduce tumorigenesis and aberrant development associated with high functional levels of TAg present in vivo, and to concomitantly allow immortalization of cell cultures at permissive temperatures, the H-2Kb-tsA58 transgene has the interferon-inducible Class I antigen promoter from the mouse H-2Kb locus directing interferon-inducible expression of a temperature sensitive TAg to a broad range of tissues. The tsA58 TAg gene product is functional at the permissive temperature of 33-34.5 degrees C. (33-34.5° C.) but is rapidly denatured at the nonpermissive temperature of 39.5 degrees C. (39.5° C. The AVIC Cell line established from the adult aortic valves of H-2Kb-tsA58 mice is conditionally immortal in their growth: cells can be grown for extended periods in vitro simply by growing them at 33 Degrees Celsius in the presence of interferon-γ, while still retaining the capacity to undergo normal VIC differentiation in vitro. Importantly, osteogenic media can be used to induce osteogenic differentiation and calcification of the AVIC cells in vitro.

The Role of Mohamad Azhar, PhD (Principal Investigator and Inventor)

Dr. Azhar contributed to the conceptualization, methodology, formal analysis, writing (review and editing of the disclosure), supervision of Dr. Mrinmay Chakrabarti, and funding Acquisition.

The Role of Mrinmay Chakrabarti, PhD (Postdoctoral Fellow)

Dr. Chakrabarti is a currently a postdoctoral fellow employed by Dr. Azhar. He receives direct instructions from Dr. Azhar. Dr. Chakrabarti contributed to the methodology. Briefly, Dr. Chakrabarti collected aortic tissue and performed cell culture experiments.

SEQUENCE LISTING - USC 2033101.000310
<110> University of South Carolina
<120> IN VITRO ASSAY FOR DRUG DISCOVERY OF
VALVULAR HEART DISEASE
<130> 2033101.0000310
<140> Unknown
<141> Sep. 14, 2021
<150> U.S. Provisional Application No. 63/077,837
<151> Sep. 14, 2020
<160> 1
<170> PatentIn
<210> 1
<211> 369
<212> PRT
<213> Mus musculus
<221> CDS
<222> 1 . . . 369
<400> 1
Met Val Pro Cys Thr Leu Leu Leu Leu Leu Ala Ala
Ala Leu Ala Pro
Thr Gln Thr Arg Ala Gly Pro His Ser Leu Arg Tyr
Phe Val Thr Ala
Val Ser Arg Pro Gly Leu Gly Glu Pro Arg Tyr Met
Glu Val Gly Tyr
Val Asp Asp Thr Glu Phe Val Arg Phe Asp Ser Asp
Ala Glu Asn Pro
Arg Tyr Glu Pro Arg Ala Arg Trp Met Glu Gln Glu
Gly Pro Glu Tyr
Trp Glu Arg Glu Thr Gln Lys Ala Lys Gly Asn Glu
Gln Ser Phe Arg
Val Asp Leu Arg Thr Leu Leu Gly Tyr Tyr Asn Gln
Ser Lys Gly Gly
Ser His Thr Ile Gln Val Ile Ser Gly Cys Glu Val
Gly Ser Asp Gly
Arg Leu Leu Arg Gly Tyr Gln Gln Tyr Ala Tyr Asp
Gly Cys Asp Tyr
Ile Ala Leu Asn Glu Asp Leu Lys Thr Trp Thr Ala
Ala Asp Met Ala
Ala Leu Ile Thr Lys His Lys Trp Glu Gln Ala Gly
Glu Ala Glu Arg
Leu Arg Ala Tyr Leu Glu Gly Thr Cys Val Glu Trp
Leu Arg Arg Tyr
Leu Lys Asn Gly Asn Ala Thr Leu Leu Arg Thr Asp
Ser Pro Lys Ala
His Val Thr His His Ser Arg Pro Glu Asp Lys Val
Thr Leu Arg Cys
Trp Ala Leu Gly Phe Tyr Pro Ala Asp Ile Thr Leu
Thr Trp Gln Leu
Asn Gly Glu Glu Leu Ile Gln Asp Met Glu Leu Val
Glu Thr Arg Pro
Ala Gly Asp Gly Thr Phe Gln Lys Trp Ala Ser Val
Val Val Pro Leu
Gly Lys Glu Gln Tyr Tyr Thr Cys His Val Tyr His
Gln Gly Leu Pro
Glu Pro Leu Thr Leu Arg Trp Glu Pro Pro Pro Ser
Thr Val Ser Asn
Met Ala Thr Val Ala Val Leu Val Val Leu Gly Ala
Ala Ile Val Thr
Gly Ala Val Val Ala Phe Val Met Lys Met Arg Arg
Arg Asn Thr Gly
Gly Lys Gly Gly Asp Tyr Ala Leu Ala Pro Gly Ser
Gln Thr Ser Asp
Leu Ser Leu Pro Asp Cys Lys Val Met Val His Asp
Pro His Ser Leu
Ala

Various modifications and variations of the described methods, pharmaceutical compositions, and kits of the disclosure will be apparent to those skilled in the art without departing from the scope and spirit of the disclosure. Although the disclosure has been described in connection with specific embodiments, it will be understood that it is capable of further modifications and that the disclosure as claimed should not be unduly limited to such specific embodiments. Indeed, various modifications of the described modes for carrying out the disclosure that are obvious to those skilled in the art are intended to be within the scope of the disclosure. This application is intended to cover any variations, uses, or adaptations of the disclosure following, in general, the principles of the disclosure and including such departures from the present disclosure come within known customary practice within the art to which the disclosure pertains and may be applied to the essential features herein before set forth.

What is claimed is:

1. A cell culture-based valve calcification assay comprising:
at least one valve interstitial cell;
wherein the at least one valve interstitial cell is derived from at least one Ts(H2-K1-tsA58) mouse;
wherein at least one compound is exposed to the at least one valve interstitial cell; and
evaluating the at least one valve interstitial cell to determine if the at least one compound inhibited osteogenic differentiation or calcification in the at least one valve interstitial cell.

2. The cell culture-based valve calcification assay of claim 1, wherein osteogenic differentiation is promoted via separating at least two valve interstitial cells onto at least two separate media wherein at least one media is osteogenic.

3. The cell culture-based valve calcification assay of claim 1, wherein evaluating occurs via staining the at least one valve interstitial cell.

4. The cell culture-based valve calcification assay of claim 3, wherein an extent of staining is quantified to evaluate effectiveness of the at least one compound.

5. The cell culture-based valve calcification assay of claim 1, wherein the assay is used to identify at least one molecular mechanism or pathway involved in the pathogenesis of valvular heart disease including aortic valve stenosis, myxomatous mitral valve disease, or mitral valve prolapse.

6. The cell culture-based valve calcification assay of claim 1, wherein the at least one valve interstitial cell exhibits a pathological process involved in aortic valve and mitral valve calcification.

7. A method of identifying new compounds for treatment of valve calcification comprising:
introducing at least one potential treatment compound to an assay;
wherein the assay contains at least one valve interstitial cell derived from at least one mouse cell;
wherein the at least one valve interstitial cell is obtained from at least one Ts(H2-K1-tsA58) mouse and exhibits a pathological processes causing aortic valve and mitral valve calcification; and
evaluating the at least one valve interstitial cell to determine if the at least one potential treatment compound inhibited osteogenic differentiation or calcification in the at least one valve interstitial cell.

8. The method of claim 7, wherein the at least one potential treatment compound is a novel drug, molecule, chemical, or small molecule inhibitor.

9. The method of claim 7, further comprising evaluating via staining the at least one valve interstitial cell.

10. The method of claim 9, wherein an extent of staining is quantified to evaluate effectiveness of the at least one potential treatment compound.

11. The method of claim 7, wherein the assay is used to identify at least one molecular mechanism or pathway involved in the pathogenesis of valvular heart disease including aortic valve stenosis, myxomatous mitral valve disease, or mitral valve prolapse.

12. A method for obtaining a novel, stable line of valve interstitial cells comprising:
collecting at least one aortic valve leaflet from at least one Ts(H2-K1-tsA58) mouse;
centrifuging the at least one aortic valve leaflet;
digesting the at least one aortic valve leaflet to remove valvular endothelial cells;
pelleting the at least one aortic valve leaflet by centrifugation;
washing and repelleting the at least one aortic valve leaflet; and
harvesting at least one valve interstitial cell following digestion.

13. The method of claim 12, wherein the stable line of valve interstitial cells allows inducible expression of at least one thermolabile large tumor antigen.

14. The method of claim 12, wherein tumorigenesis in the stable line of valve interstitial cells is temperature dependent and tumorigenesis is inhibited at a first select temperature but allowed at a second select temperature.
